(19) United States
(12) Patent Application Publication
(10) Pub. No.: US 2009/0304754 A1
(43) Pub. Date: Dec. 10, 2009

(54) Pegylated glutenase polypeptides

(76) Inventor: Srebrenka Robic, Decatur, GA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/089,592

(22) PCT Filed: Oct. 11, 2006
(86) PCT No.: PCT/US06/39714

§ 371 Date: Aug. 5, 2008

Related U.S. Application Data
(60) Provisional application No. 60/726,130, filed on Oct. 12, 2005.

(51) Int. Cl. A61K 9/00; C12N 9/50; C12N 9/52; A61K 38/48; A61P 1/00

(52) U.S. Cl. 424/400; 435/219; 435/220; 424/94.63

(57) ABSTRACT

Glutenase proteins, such as prolyl endopeptidases, are stabilized by covalent PEG modification.

### BACKGROUND OF THE INVENTION

In 1953, it was first recognized that ingestion of gluten, a common dietary protein present in wheat, barley and rye causes disease, now called Celiac sprue, in sensitive individuals. Gluten is a complex mixture of glutamine- and proline-rich glutenin and prolamine molecules, which is thought to be responsible for disease induction. Ingestion of such proteins by sensitive individuals produces flattening of the normally luxurious, rug-like, epithelial lining of the small intestine known to be responsible for efficient and extensive terminal digestion of peptides and other nutrients. Clinical symptoms of Celiac Sprue include fatigue, chronic diarrhea, malabsorption of nutrients, weight loss, abdominal distension, anemia, as well as a substantially enhanced risk for the development of osteoporosis and intestinal malignancies (lymphoma and carcinoma). The disease has an incidence of approximately 1 in 100 in European populations.

A related disease is dermatitis herpetiformis, which is a chronic eruption characterized by clusters of intensely pruritic vesicles, papules, and urticaria-like lesions. IgA deposits occur in almost all normal-appearing and perilesional skin. Asymptomatic gluten-sensitive enteropathy is found in 75 to 90% of patients and in some of their relatives. Onset is usually gradual. Itching and burning are severe, and scratching often obscures the primary lesions with eczematization of nearby skin, leading to an erroneous diagnosis of eczema. Strict adherence to a gluten-free diet for prolonged periods may control the disease in some patients, obviating or reducing the requirement for drug therapy. Dapsone, sulfapyridine and colchicines are sometimes prescribed for relief of itching.

Celiac Sprue is generally considered to be an autoimmune disease and the antibodies found in the serum of the patients supports a theory of an immunological nature of the disease. Antibodies to tissue transglutaminase (tTG) and gliadin appear in almost 100% of the patients with active Celiac Sprue, and the presence of such antibodies, particularly of the IgA class, has been used in diagnosis of the disease.

The large majority of patients express the HLA-DQ2 [DQ(a1*0501, b1*02)] and/or DQ8 [DQ(a1*0301, b1*0302)] molecules. It is believed that intestinal damage is caused by interactions between specific gliadin oligopeptides and the HLA-DQ2 or DQ8 antigen, which in turn induce proliferation of T lymphocytes in the sub-epithelial layers. T helper 1 cells and cytokines apparently play a major role in a local inflammatory process leading to villus atrophy of the small intestine.

At the present time there is no good therapy for the disease, except to completely avoid all foods containing gluten. Although gluten withdrawal has transformed the prognosis for children and substantially improved it for adults, some people still die of the disease, mainly adults who had severe disease at the outset. An important cause of death is iymphoreticular disease (especially intestinal lymphoma). It is not known whether a gluten-free diet diminishes this risk. Apparent clinical remission is often associated with histologic relapse that is detected only by review biopsies or by increased EMA titers.

Gluten is so widely used, for example in commercial soups, sauces, ice creams, hot dogs, and other foods, that patients need detailed lists of foodstuffs to avoid and expert advice from a dietitian familiar with celiac disease. Ingesting even small amounts of gluten may prevent remission or induce relapse. Supplementary vitamins, minerals, and hematinics may also be required, depending on deficiency. A few patients respond poorly or not at all to gluten withdrawal, either because the diagnosis is incorrect or because the disease is refractory. In the latter case, oral corticosteroids (e.g., prednisone 10 to 20 mg bid) may induce response.

A promising new therapy in development involves the oral administration of a protease or mixture of proteases that, together with endogenous enzymes of the stomach and small intestine, can degrade gluten to amino acids and small peptides unable to induce the autoimmune response in sensitive individuals. Such therapies and proteases useful in their practice are described in PCT patent publications 2005/107786 and 2003/0215438, incorporated herein by reference. However, the harsh conditions of the stomach and small intestine can degrade such proteases, and methods and reagents for stabilizing them to make the therapies more effective, both in treatment results and in cost of treatment, are needed.

In view of the serious and widespread nature of Celiac Sprue, improved methods of treating or ameliorating the effects of the disease are needed. The present invention addresses such needs.

### SUMMARY OF THE INVENTION

The present invention provides compositions and methods for treating the symptoms of Celiac Sprue and/or dermatitis herpetiformis by decreasing the levels of toxic gluten oligopeptides in foodstuffs. The present invention relates to the discovery that glutenases are stabilized for enteric delivery by covalent addition of polyethylene glycol to the glutenase, a process termed “PEGylation”, and that PEGylation can increase the relative activity of the enzyme against gluten oligopeptides and in any event makes the PEGylated glutenase more resistant to degradation under physiological conditions.

In one aspect, the present invention provides physiologically more stable, modified glutenases for in vivo use in the detoxification of gluten. The invention also provides methods for making such modified glutenases. In one method of the invention, an active glutenase or a non-denatured proenzyme form of the glutenase is coupled to a modification reagent under conditions such that coupling occurs primarily or exclusively at the surface of the protein. In one embodiment, the surface-modified glutenases of the invention are modified by PEGylation. In other embodiments, the method of modifying the protein surfaces utilizes another suitable modification reagent that will stabilize the protease to physiological conditions without rendering it inactive. Such other reagents include but are not limited to those employed in methods such as acylation (e.g. Kurtzhals et al, Biochem J. 312, 725-731, 1995; Foldvari et al, J. Pharm Sci 87, 1203-1208, 1998; Knudsen et al, J. Med Chem 43, 1664-1669, 2000) and glycosylation (e.g. Kim et al, Biochem. Biphys. Res. Cummun. 315(4):976-83, 2004; Pratam et al Appl Microbiol Biotechnol. 53(4):469-75, 2000).

In one embodiment of the invention, a PEGylated glutenase is administered to a patient and acts internally to destroy the toxic oligopeptides. Compositions and methods for the administration of enteric formulations of one or more PEGylated glutenases, each of which may be present as a single agent or a combination of active agents are provided. Such formulations include formulations in which the PEGylated glutenase is contained within an enteric coating that allows delivery of the active agent to the intestine and formulations in which the active agents are stabilized to resist digestion in acidic stomach conditions.

In one embodiment of the invention, the PEGylated glutenase is a bacterial prolyl endopeptidase or variant derived therefrom. In other embodiments, the PEGylated glutenase is one or more enzymes from Flavobacterium meningosepticum (FM), Sphingomonas capsulata (SC) and Myxococcus xanthus (MX). The enzymes exhibit differences in activity profile with respect to chain length and subsite specificity. In one embodiment of the invention, one or more of the FM; SC and MX PEPs, where at least one enzyme is PEGylated, are used to decrease the levels of toxic gluten oligopeptides in foodstuffs. In another embodiment of the invention, one or more of these proteases or another protease active in the small intestine is co-administered with another PEP, including but not limited to the PEP derived from Aspergillus niger described in US patent application publication No. 2004-0241664-A1, or other protease, such as the barley cysteine proteinase B, that is active in the stomach.

In some embodiments, the invention provides a PEGylated glutenase, as well as pharmaceutical formulations of a PEGylated glutenase. Such formulations include, without limitation, capsules, pills, and the like, which optionally comprise an enteric coating; as well as sachets, powders, and the like. In another aspect, the invention provides pharmaceutical formulations containing one or more PEGylated glutenases and a pharmaceutically acceptable carrier. Such formulations include formulations in which the glutenase is contained within an enteric coating that allows delivery of the active agent to the intestine and formulations in which the active agents are otherwise stabilized to resist digestion in acidic stomach conditions. The formulation may comprise one or more glutenases or a mixture or “cocktail” of agents having different activities. Depending upon their pH optima, glutenases can hydrolyze gluten or gluten peptides in the stomach (i.e. at strongly acidic pH values) or in the small intestine (i.e. mildly acidic pH values).

In another aspect, the invention provides methods for treating Celiac Sprue by administering a PEGylated glutenase. In one embodiment, the glutenase is administered orally. In one embodiment, at least 10 mg of pegylated glutenase is administered, where the weight is the protein weight prior to pegylation. In other embodiments, at least 100 mg, 250 mg, 500 mg or more of glutenase are administered, where the weight is the protein weight prior to pegylation. In one embodiment, sufficient glutenase to hydrolyze at least 1 g of gluten is administered. In other embodiments, sufficient glutenase is administered to hydrolyze 5 g, 10 g, 20 g or more gluten is administered.

These and other aspects and embodiments of the invention are described in more detail below.

### BRIEF DESCRIPTION OF THE DRAWINGS

FIG. 1. SDS-PAGE gel of PEGylated PEPs. (1) MW Marker, (2) Unmodified FM PEP, (3) FM PEG-2000, (4) FM PEG 5000, (5) FM PEG-20,000, (6) FM PEG-30,000, (7) unmodified MX PEP, (8) MX PEG-2000, (9) MX PEG-5000, (10) MX PEG-20,000, (11) MX PEG-30,000.

FIG. 2. HPLC-monitored time-course of digestion of 26mer peptide by the native FM PEP (a), FMPEP-5k (b) and FMPEP-20k (c).

FIG. 3. Dependence of the rate of FM PEP degradation by trypsin (a) and chymotrypsin (b) on concentration of FM PEP. Comparison between unmodified (black circles) and FM PEP conjugated with 20 k PEG (squares).

### DETAILED DESCRIPTION OF THE EMBODIMENTS

Polypeptides delivered orally are susceptible to various degradative conditions, including proteolytic digestion in the presence of enzymes in the stomach and small intestine and bile salts in the intestine. The resistance of glutenases to proteolytic degradation generally and enteric degradation in particular is increased by PEGylation. PEGylated proteases and pharmaceutical formulations for this purpose are provided.

The present invention relates generally to methods and reagents useful in formulating polypeptides for oral administration, particularly where enteric delivery is desirable. Thus, the practice of the present invention may employ conventional techniques of molecular biology (including recombinant techniques), microbiology, cell biology, biochemistry, peptide chemistry and immunology within the scope of those of skill in the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology” (D. M. Weir & C. C. Blackwell, eds.); “Gene Transfer Vectors for Mammalian Cells” (J. M. Miller & M. P. Calos, eds., 1987); “Current Protocols in Molecular Biology” (F. M. Ausubel et al., eds., 1987); “PCR: The Polymerase Chain Reaction” (Mullis et al., eds., 1994); and “Current Protocols in Immunology” (J. E. Coligan et al., eds., 1991); as well as updated or revised editions of all of the foregoing.

Methods and compositions are provided for the administration of one or more PEGylated glutenases to a patient suffering from Celiac Sprue and/or dermatitis herpetiformis. In some patients, these methods and compositions will allow the patient to ingest glutens without serious health consequences, much the same as individuals that do not suffer from either of these conditions. In some embodiments, the formulations of the invention comprise a PEGylated glutenase contained in an enteric coating that allows delivery of the active agent(s) to the intestine; in other embodiments, the active agent(s) is stabilized to resist digestion in acidic stomach conditions. In some cases the active agent(s) have hydrolytic activity under acidic pH conditions, and can therefore initiate the proteolytic process on toxic gluten sequences in the stomach itself.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease; (b) inhibiting the disease, i.e., arresting its development; and (c) relieving the disease, i.e., causing regression of the disease.

The terms “individual,” “host,” “subject,” and “patient” are used interchangeably herein, and refer to a mammal, including, but not limited to, primates and humans.

The present invention relates generally to methods and reagents useful in treating foodstuffs containing gluten with enzymes that digest the oligopeptides toxic to Celiac Sprue patients. Although specific enzymes are exemplified herein, any of a number of alternative enzymes and methods apparent to those of skill in the art upon contemplation of this disclosure are equally applicable and suitable for use in practicing the invention. The methods of the invention, as well as tests to determine their efficacy in a particular patient or application, can be carried out in accordance with the teachings herein using procedures standard in the art. Thus, the practice of the present invention may employ conventional techniques of molecular biology (including recombinant techniques), microbiology, cell biology, biochemistry and immunology within the scope of those of skill in the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology” (D. M. Weir & C. C. Blackwell, eds.); “Gene Transfer Vectors for Mammalian Cells” (J. M. Miller & M. P. Calos, eds., 1987); “Current Protocols in Molecular Biology” (F. M. Ausubel et al., eds., 1987); “PCR: The Polymerase Chain Reaction” (Mullis et al., eds., 1994); and “Current Protocols in Immunology” (J. E. Coligan et al., eds., 1991); as well as updated or revised editions of all of the foregoing.

As used herein, the term “glutenase” refers to an enzyme useful in the methods of the present invention that is capable, alone or in combination with endogenous or exogenously added enzymes, of cleaving toxic oligopeptides of gluten proteins of wheat, barley, oats and rye into non-toxic fragments. For example, see US patent application publication Nos. US-2003-0215438-A1 US-2005-0249719-A1 and PCT patent publication 2005/107786, each herein specifically incorporated by reference. Gluten is the protein fraction in cereal dough, which can be subdivided into glutenins and prolamines, which are subclassified as gliadins, secalins, hordeins, and avenins from wheat, rye, barley and oats, respectively. For further discussion of gluten proteins, see the review by Wieser (1996) Acta Paediatr Suppl. 412:3-9, incorporated herein by reference.

In one embodiment, the term “glutenase” as used herein refers to a protease or a peptidase enzyme that meets one or more of the criteria provided herein. Using these criteria, one of skill in the art can determine the suitability of a candidate enzyme for use in the methods of the invention. Many enzymes will meet multiple criteria, including two, three, four or more of the criteria, and some enzymes will meet all of the criteria. The terms “protease” or “peptidase” can refer to a glutenase and as used herein describe a protein or fragment thereof with the capability of cleaving peptide bonds, where the scissile peptide bond may either be terminal or internal in oligopeptides or larger proteins. Prolyl-specific peptidases are glutenases useful in the practice of the present invention.

Glutenases of the invention include protease and peptidase enzymes having at least about 20% sequence identity at the amino acid level, more usually at least about 40% sequence identity, and preferably at least about 70% sequence identity to one of the following peptidases: prolyl endopeptidase (PEP) from F. meningosepticum (Genbank accession number D10980), PEP from A. hydrophila (Genbank accession number D14005), PEP form S. capsulata (Genbank accession number AB010298), DCP I from rabbit (Genbank accession number X62551), PEP from Aspergillus niger, DPP IV from Aspergillus fumigatus (Genbank accession number U87950), and cysteine proteinase B from Hordeum vulgare (Genbank accession number JQ1110).

Each of the above proteases described herein can be engineered to improve desired properties such as enhanced specificity toward toxic gliadin sequences, improved tolerance for longer substrates, acid stability, pepsin resistance, resistance to proteolysis by the pancreatic enzymes and improved shelf-life. The desired property can be engineered via standard protein engineering methods.

In one embodiment of the present invention, the glutenase is a PEP. Homology-based identification (for example, by a PILEUP sequence analysis) of prolyl endopeptidases can be routinely performed by those of skill in the art upon contemplation of this disclosure to identify PEPs suitable for use in the methods of the present invention. PEPs are produced in microorganisms, plants and animals. PEPs belong to the serine protease superfamily of enzymes and have a conserved catalytic triad composed of a Ser, His, and Asp residues. Some of these homologs have been characterized, e.g. the enzymes from F. meningoscepticum, Aspergillus niger, Aeromonas hydrophila, Aeromonas punctata, Novosphingobium capsulatum, Pyrococcus furiosus and from mammalian sources are biochemically characterized PEPs. Others such as the Nostoc and Arabidopsis enzymes are likely to be PEPs but have not been fully characterized to date. Homologs of the enzymes of interest may be found in publicly available sequence databases, and the methods of the invention include such homologs. Candidate enzymes are expressed using standard heterologous expression technologies, and their properties are evaluated using the assays described herein.

In one embodiment of the invention, the glutenase is Flavobacterium meningosepticum PEP (Genbank ID # D10980). Relative to the F. meningoscepticum enzyme, the pairwise sequence identity of this family of enzymes is in the 30-60% range. Accordingly, PEPs include enzymes having >30% identity to the F. meningoscepticum enzyme (as in the Pyrococcus enzymes), or having >40% identity (as in the Novosphingobium enzymes), or having >50% identity (as in the Aeromonas enzymes) to the F. meningoscepticum enzyme. A variety of assays have verified the therapeutic utility of this PEP. In vitro, this enzyme has been shown to rapidly cleave several toxic gluten peptides, including the highly inflammatory 33-mer, (SEQ ID NO:12) LQLQPFPQPQLPYPQPQLPYPQPQLPYPQPQPF. In vivo it acts synergistically with the peptidases of the intestinal brush border membrane so as to rapidly detoxify these peptides, as well as gluten that has been pre-treated with gastric and pancreatic proteases. It has broad chain length specificity, making it especially well suited for the breakdown of long proline-rich peptides released into the duodenum from the stomach. The enzyme has a pH optimum around pH 7, and has high specific activity under conditions that mimic the weakly acidic environment of the upper small intestine. Flavobacterium PEP can cleave all T cell epitopes in gluten that have been tested to date. It has particular preference for the immunodominant epitopes found in alpha-gliadin. When grocery-store gluten is treated with this PEP, a rapid decrease in its antigenicity can be observed, as judged by LC-MS analysis and testing against polyclonal T cell lines derived from small intestinal biopsies from Celiac Sprue patients. The denatured protein is non-allergenic in rodents, rabbits and humans. It is relatively stable toward destruction by pancreatic proteases, an important feature since under physiological conditions it will be expected to act in concert with those enzymes.

Another enzyme of interest is Myxococcus xanthus PEP (Genbank ID# AF127082), which is provided in PEGylated form by the present invention. This enzyme possesses many of the advantages of the Flavobacterium PEP. It can cleave the 33-mer into small non-toxic peptides. Whereas the Flavobacterium enzyme appears to have a relatively strict preference for PQ bonds in gliadin peptides, the Myxococcus enzyme can cleave at PQ, PY and PF bonds, a feature that allows it to proteolyze a broader range of gluten epitopes. Compared to the Flavobacterium enzyme, it has equivalent stability toward the pancreatic proteases and superior stability toward acidic environments. The Myxococcus enzyme is well expressed in E. coli, making it feasible to produce this enzyme cost-effectively.

Another enzyme of interest is Sphingomonas capsulata PEP (Genbank ID# AB010298), which is provided in PEGylated form by the present invention. This enzyme is comparable to the Flavobacterium and Myxococcus enzymes. It has broader sequence and pH specificity than either the Flavobacterium or the Myxococcus PEP, and may therefore be able to destroy the widest range of antigenic epitopes, while also being active in the stomach. Like the Myxococcus enzyme, it is also well expressed in E. coli.

Another enzyme of interest is Lactobacillus helveticus PEP (Genbank ID# 321529), which is provided in PEGylated form by the present invention. Unlike the above PEPs, this PEP is a zinc enzyme. It can efficiently proteolyze long peptide substrates such as the casein peptides (SEQ ID NO:28) YQEPVLGPVRGPFPIIV and (SEQ ID NO:29) RPKHPIKHQ. Proteolysis occurs at all PV and PI subsites, suggesting the PEP prefers hydrophobic residues at the S1′ position, as are frequently found in gluten. Because the producer strain of L. helveticus CNRz32 is commonly used in cheesemaking, this enzyme has desirable properties as a food-grade enzyme.

Another enzyme of interest is Penicillium citrinum PEP (Genbank ID# D25535), which is provided in PEGylated form by the present invention. This enzyme has been shown to possess PEP activity based on its ability to cleave a number of Pro-Xaa bonds effectively in peptides such as dynorphin A and substance P. The putative metalloprotease has the advantages of small size and a pH profile that renders it suitable to working in concert with the pancreatic enzymes in the duodenum. As such, it can be used to detoxify gluten for the treatment of Celiac Sprue.

Other than proline, glutamine residues are also highly prevalent in gluten proteins. The toxicity of gluten in Celiac Sprue has been directly correlated to the presence of specific Gln residues. Therefore, glutamine-specific proteases are also beneficial for the treatment of Celiac Sprue. Because oats contain proteins that are rich in glutamine but not especially rich in proline residues, an additional benefit of a glutamine-specific protease is the improvement of oat tolerance in those celiac patients who show mild oat-intolerance. An example of such a protease is the above-mentioned cysteine endoproteinase from Hordeum vulgare endoprotease (Genbank accession U19384), and the present invention provides this enzyme in PEGylated form. This enzyme cleaves gluten proteins rapidly with a distinct preference for post-Gln cleavage. The enzyme is active under acidic conditions, and is useful as an orally administered dietary supplement. A gluten-containing diet may be supplemented with orally administered proEPB2, resulting in effective degradation of immunogenic gluten peptides in the acidic stomach, before these peptides enter the intestine and are presented to the immune system. The proEPB2 is the zymogen form of the Hordeum vulgare EPB2 protease; the acidic conditions of the stomach activate the zymogen; the present invention provides PEGylated forms of both the proEPB2 and EPB2 enzymes. Proteins with high sequence similarity to this enzyme are also of interest and PEGylated versions of them are provided by the present invention. An advantage of these enzymes is that they are considered as safe for human oral consumption, due to their presence in dietary gluten from barley.

Intestinal dipeptidyl peptidase IV and dipeptidyl carboxypeptidase I are the rate-limiting enzymes in the breakdown of toxic gliadin peptides from gluten. These enzymes are bottlenecks in gluten digestion in the mammalian small intestine because (i) their specific activity is relatively low compared to other amino- and carboxy-peptidases in the intestinal brush border; and (ii) due to their strong sensitivity to substrate chain length, they cleave long immunotoxic peptides such as the 33-mer extremely slowly. Both these problems can be ameliorated through the administration of proline-specific amino- and carboxy-peptidases from other sources. For example the X-Pro dipeptidase from Aspergillus oryzae (GenBank ID# BD191984) and the carboxypeptidase from Aspergillus saitoi (GenBank ID# D25288) can improve gluten digestion in the Celiac intestine. PEGylated forms of these enzymes are provided by the present invention.

The glutenase proteins of the present invention may be prepared by in vitro synthesis, using conventional methods as known in the art. Various commercial synthetic apparatuses are available, for example, automated synthesizers by Applied Biosystems, Inc., Foster City, Calif., Beckman, and other manufacturers. Using synthesizers, one can readily substitute for the naturally occurring amino acids one or more unnatural amino acids. The particular sequence and the manner of preparation will be determined by convenience, economics, purity required, and the like. If desired, various groups can be introduced into the protein during synthesis that allow for linking to other molecules or to a surface. For example, cysteines can be used to make thioethers, histidines can be used for linking to a metal ion complex, carboxyl groups can be used for forming amides or esters, amino groups can be used for forming amides, and the like.

The glutenase proteins useful in the practice of the present invention may also be isolated and purified in accordance with conventional methods from recombinant production systems and from natural sources. Protease production can be achieved using established host-vector systems in organisms such as E. coli, S. cerevisiae, P. pastoris, Lactobacilli, Bacilli and Aspergilli. Integrative or self-replicative vectors may be used for this purpose. In some of these hosts, the protease is expressed as an intracellular protein and subsequently purified, whereas in other hosts the enzyme is secreted into the extracellular medium. Purification of the protein can be performed by a combination of ion exchange chromatography, Ni-affinity chromatography (or some alternative chromatographic procedure), hydrophobic interaction chromatography, and/or other purification techniques. Typically, the compositions used in the practice of the invention will comprise at least 20% by weight of the desired product, more usually at least about 75% by weight, preferably at least about 95% by weight, and for therapeutic purposes, usually at least about 99.5% by weight, in relation to contaminants related to the method of preparation of the product and its purification. Usually, the percentages will be based upon total protein.

### PEGylated Glutenase

The term PEGylated glutenase as used herein refers to derivatives of glutenase that are chemically modified with one or more polyethylene glycol moieties, i.e., PEGylated. The PEG molecule of a PEGylated glutenase is conjugated to one or more amino acid side chains of the glutenase. In some embodiments, the PEGylated glutenase contains a PEG moiety on only one amino acid. In other embodiments, the PEGylated glutenase contains a PEG moiety on two or more amino acids, e.g., the glutenase contains a PEG moiety attached to two or more, five or more, ten or more, fifteen or more, or twenty or more different amino acid residues. In some embodiments, the PEG chain is 2000, greater than 2000, 5000, greater than 5,000, 10,000, greater than 10,000, greater than 10,000, 20,000, greater than 20,000, and 30,000 Da.

The polypeptide may be coupled directly to PEG (i.e., without a linking group) through an amino group, a sulfhydryl group, a hydroxyl group, or a carboxyl group.

The synthetic methods provided by the invention are sufficiently varied that one can make a wide variety of PEGylated glutenases. The various forms provided can vary, for example, with respect to the size and composition of the PEG and the site and nature of the covalent linkage between the PEG and the glutenase. For example, any one or any combination of the amino acids in a glutenase can be modified. For example, in some embodiments, the PEGylated glutenase might be PEGylated at or near the amino terminus (N-terminus) of the glutenase polypeptide, e.g., the PEG moiety is conjugated to the glutenase polypeptide at one or more amino acid residues from amino acid 1 through amino acid 4, or from amino acid 5 through about 10. In other embodiments, the PEGylated glutenase might be PEGylated at or near the carboxyl terminus (C-terminus) of the glutenase polypeptide. In other embodiments, the PEGylated glutenase might be PEGylated at one or more internal amino acid residues.

In some embodiments, PEG is attached to the glutenase via a linking group. The linking group is any biocompatible linking group, where “biocompatible” indicates that the compound or group is non-toxic and may be utilized in vitro or in vivo without causing injury, sickness, disease, or death. PEG can be bonded to the linking group, for example, via an ether bond, an ester bond, a thiol bond or an amide bond. Suitable biocompatible linking groups include, but are not limited to, an ester group, an amide group, an imide group, a carbamate group, a carboxyl group, a hydroxyl group, a carbohydrate, a succinimide group (including, for example, succinimidyl succinate (SS), succinimidyl propionate (SPA), succinimidyl butanoate (SBA), succinimidyl carboxymethylate (SCM), succinimidyl succinamide (SSA) or N-hydroxy succinimide (NHS)), an epoxide group, an oxycarbonylimidazole group (including, for example, carbonyldimidazole (CDI)), a nitro phenyl group (including, for example, nitrophenyl carbonate (NPC) or trichlorophenyl carbonate (TPC)), a trysylate group, an aldehyde group, an isocyanate group, a vinylsulfone group, a tyrosine group, a cysteine group, a histidine group or a primary amine. If an intact, properly folded glutenase protein is reacted with the PEG coupling reagent, then the PEG groups will preferentially react with surface residues as opposed to buried residues, which provides practical, cost-efficent procedures for protein PEGylation and synthesis of the PEGylated glutenases of the invention. For example, as illustrated in Experimental Section below, surface lysines of two PEPs can be PEGylated to completion without loss of activity.

Methods for making succinimidyl propionate (SPA) and succinimidyl butanoate (SBA) ester-activated PEGs are described in U.S. Pat. No. 5,672,662 (Harris, et al.) and WO 97/03106.

Methods for attaching a PEG to a polypeptide are known in the art, and any known method can be used in accordance with the methods of the invention to produce a PEGylated glutenase of the invention. See, for example, by Park et al, Anticancer Res., 1:373-376 (1981); Zaplipsky and Lee, Polyethylene Glycol Chemistry: Biotechnical and Biomedical Applications, J. M. Harris, ed., Plenum Press, NY, Chapter 21 (1992); U.S. Pat. No. 5,985,265; U.S. Pat. No. 5,672,662 (Harris, et al.) and WO 97/03106.

In many embodiments, the PEG is a monomethoxy PEG molecule that reacts with primary amine groups on the glutenase. Methods of modifying polypeptides with monomethoxy PEG via reductive alkylation are known in the art. See, e.g., Chamow et al. (1994) Bioconj. Chem. 5:133-140.

Polyethylene glycol. Polyethylene glycol suitable for conjugation to a glutenase is soluble in water at room temperature, and has the general formula R(O—CH₂—CH₂)ₙO—R, where R is hydrogen or a protective group such as an alkyl or an alkanol group, and where n is an integer from 1 to 1000. Where R is a protective group, it generally has from 1 to 8 carbons.

In many embodiments, PEG has at least one hydroxyl group, e.g., a terminal hydroxyl group, which hydroxyl group is modified to generate a functional group that is reactive with an amino group, e.g., an epsilon amino group of a lysine residue, a free amino group at the N-terminus of a polypeptide, or any other amino group such as an amino group of asparagine, glutamine, arginine, or histidine, to facilitate covalent modification of a polypeptide with PEG.

In other embodiments, PEG is derivatized so that it is reactive with free carboxyl groups in the glutenase. Suitable derivatives of PEG that are reactive with the free carboxyl group at the carboxyl-terminus of glutenase include, but are not limited to PEG-amine, and hydrazine derivatives of PEG (e.g., PEG-NH—NH₂).

In other embodiments, PEG is derivatized such that it comprises a terminal thiocarboxylic acid group, —COSH, which selectively reacts with amino groups to generate amide derivatives. Because of the reactive nature of the thio acid, selectivity of certain amino groups over others is achieved. For example, —SH exhibits sufficient leaving group ability in reaction with N-terminal amino group at appropriate pH conditions such that the ε-amino groups in lysine residues are protonated and remain non-nucleophilic. On the other hand, reactions under suitable pH conditions may make some of the accessible lysine residues react with selectivity.

In other embodiments, the PEG comprises a reactive ester such as an N-hydroxy succinimidate at the end of the PEG chain. Such an N-hydroxysuccinimidate-containing PEG molecule reacts with select amino groups at particular pH conditions such as neutral 6.5-7.5. For example, the N-terminal amino groups may be selectively modified under neutral pH conditions. However, if the reactivity of the reagent were extreme, accessible-NH₂ groups of lysine may also react.

In some embodiments, the PEG conjugated to the glutenase polypeptide is linear. In other embodiments, the PEG conjugated to the glutenase polypeptide is branched. Branched PEG derivatives such as those described in U.S. Pat. No. 5,643,575, “star-PEG's” and multi-armed PEG's such as those described in Shearwater Polymers, Inc. catalog “Polyethylene Glycol Derivatives 1997-1998.” Star PEGs are described in the art including, e.g., in U.S. Pat. No. 6,046,305.

PEG having a molecular weight in a range of from about 2 kDa to about 100 kDa, is generally used, where the term “about,” in the context of PEG, indicates that in preparations of polyethylene glycol, some molecules will weigh more, some less, than the stated molecular weight. For example, PEG suitable for conjugation to glutenase has a molecular weight of from about 2 kDa to about 5 kDa, from about 5 kDa to about 10 kDa, from about 10 kDa to about 15 kDa, from about 15 kDa to about 20 kDa, from about 20 kDa to about 25 kDa, from about 25 kDa to about 30 kDa, from about 30 kDa to about 40 kDa, from about 40 kDa to about 50 kDa, from about 50 kDa to about 60 kDa, from about 60 kDa to about 70 kDa, from about 70 kDa to about 80 kDa, from about 80 kDa to about 90 kDa, or from about 90 kDa to about 100 kDa.

### Preparing Peg-Glutenase Conjugates

As discussed above, the PEG moiety can be attached, directly or via a linker, to an amino acid residue at or near the N-terminus, internally, or at or near the C-terminus of a glutenase polypeptide, or a combination thereof. Conjugation can be carried out in solution or in the solid phase.

Methods for attaching a PEG moiety to an amino acid residue at or near the N-terminus of a polypeptide are known in the art. See, e.g., U.S. Pat. No. 5,985,265. Known methods for selectively obtaining an N-terminally chemically modified protein can be applied to produce PEGylated glutenase proteins of the invention. For example, a method of protein modification by reductive alkylation which exploits differential reactivity of different types of primary amino groups (lysine versus the N-terminus) available for derivatization in a particular protein can be used in accordance with the methods of the invention to prepare a PEGylated glutenase protein of the invention. Under the appropriate reaction conditions, substantially selective derivatization of the protein at the N-terminus with a carbonyl group containing polymer is achieved. The reaction is performed at pH which allows one to take advantage of the pKₐ differences between the ε-amino groups of the lysine residues and that of the α-amino group of the N-terminal residue of the protein. By such selective derivatization, attachment of a PEG moiety to the glutenase is controlled: the conjugation with the polymer takes place predominantly at the N-terminus of the glutenase, and no significant modification of other reactive groups, such as the lysine side chain amino groups, occurs.

N-terminal-specific coupling procedures such as described in U.S. Pat. No. 5,985,265 provide predominantly monoPEGylated products. The purification procedures aimed at removing the excess reagents and minor multiply PEGylated products can remove the N-terminal blocked polypeptides, and, such processes can lead to significant increases in manufacturing costs. Accordingly, the present invention also provides methods for making C-terminal PEGylated glutenase proteins and the PEGylated proteins produced as well as methods for using them to detoxify gluten in vivo. A PEG reagent that is selective for the C-terminal can be prepared with or without spacers. For example, polyethylene glycol modified as methyl ether at one end and having an amino function at the other end may be used as the starting material in the synthetic process employed to produced the PEGylated glutenase protein.

Preparing or obtaining a water-soluble carbodiimide as the condensing agent can be carried out. Coupling a glutenase with a water-soluble carbodiimide as the condensing reagent is generally carried out in aqueous medium with a suitable buffer system at an optimal pH to effect the amide linkage. A high molecular weight PEG can be added to the protein covalently to increase the molecular weight.

The selection of reagents for any particular application of the method may result from process optimization studies. A non-limiting example of a suitable reagent is EDAC or 1-ethyl-3-(3-dimethylaminopropyl) carbodiimide. The water solubility of EDAC allows for direct addition to a reaction without the need for prior organic solvent dissolution. Excess reagent and the isourea formed as the by-product of the cross-linking reaction are both water-soluble and may easily be removed by dialysis or gel filtration. A concentrated solution of EDAC in water is prepared to facilitate the addition of a small molar amount to the reaction. The stock solution is prepared and used immediately in view of the water labile nature of the reagent. Most of the synthetic protocols in literature suggest the optimal reaction medium to be in pH range between 4.7 and 6.0. However such condensation reactions do in many instances proceed without significant loss in yield even when the pH is somewhat higher than pH 6.0, such as pH of up to pH 7.5. Water may be used as solvent.

Even though the use of PEG amine has been mentioned above by name or structure, such derivatives are meant to be exemplary only, and other groups such as hydrazine derivatives as in PEG-NH—NH₂, which will also condense with the carboxyl group of the glutenase protein, can also be used. In addition to aqueous phase, the reactions can also be conducted on solid phase. Polyethylene glycol can be selected from list of compounds of molecular weight ranging from 300-40000. The choice of the various polyethylene glycols will also be dictated by the coupling efficiency and the biological performance of the purified derivative in vitro and in vivo.

Additionally, suitable spacers can be added to the C-terminal of the protein. The spacers may have reactive groups such as SH, NH₂ or COOH to couple with appropriate PEG reagent to provide the glutenase derivatives. A combined solid/solution phase methodology can be devised for the preparation of C-terminal pegylated polypeptides. For example, in one synthetic method of the invention, the C-terminus of glutenase is extended on a solid phase using a Gly-Gly-Cys-NH₂ spacer and then PEGylated in solution using activated dithiopyridyl-PEG reagent of appropriate molecular weights.

There may be a more reactive carboxyl group of amino acid residues elsewhere in the molecule that can react with the PEG reagent and lead to monoPEGylation at that site or lead to a multiply PEGylated protein of the invention, for example, a PEGylated protein in which a —COOH group in addition to the —COOH group at the C-terminus of the glutenase has been modified by PEGylation. The reaction conditions can be varied to favor or disfavor the formation of a particular type of PEGylated protein. PEGylation at a site can in some instances be minimal, such as may result from PEGylation being highly favored at another site. For example, the steric freedom at the C-terminal end of the molecule favors that site for PEGylation and so that site may be PEGylated much more favorably than another site. Alternatively, steric hindrance, such as that presented by the carbodiimide coupling agent or the structure of the PEG reagent itself, can retard or prevent PEGylation at an otherwise more reactive site.

If desired, PEGylated glutenase can be separated from unPEGylated glutenase using any known method appropriate for the purification of proteins, including, but not limited to, ion exchange chromatography, size exclusion chromatography, and combinations thereof.

In one aspect, the present invention provides a purified preparation of a PEGylated glutenase. Generally, the PEGylated glutenase species represents from about 0.5% to about 99.5% of the total population of polypeptide molecules in a population, e.g, a PEGylated glutenase species represents about 0.5%, about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or about 99.5% of the total population of polypeptide molecules in a population.

In one embodiment of the present invention, a Celiac Sprue patient is, in addition to being provided a PEGylated glutenase, provided a glutenase that is not PEGylated, an inhibitor of tissue transglutaminase, an anti-inflammatory agent, an anti-ulcer agent, a mast cell-stabilizing agents, and/or and an-allergy agent. Examples of such agents include HMG-CoA reductase inhibitors with anti-inflammatory properties such as compactin, lovastatin, simvastatin, pravastatin and atorvastatin; COX2 inhibitors such as celecoxib and rofecoxib; and p38 MAP kinase inhibitors such as BIRB-796.

As used herein, compounds which are “commercially available” may be obtained from commercial sources including but not limited to Acros Organics (Pittsburgh Pa.), Aldrich Chemical (Milwaukee Wis., including Sigma Chemical and Fluka), Apin Chemicals Ltd. (Milton Park UK), Avocado Research (Lancashire U.K.), BDH Inc. (Toronto, Canada), Bionet (Cornwall, U.K.), Chemservice Inc. (West Chester Pa.), Crescent Chemical Co. (Hauppauge N.Y.), Eastman Organic Chemicals, Eastman Kodak Company (Rochester N.Y.), Fisher Scientific Co. (Pittsburgh Pa.), Fisons Chemicals (Leicestershire UK), Frontier Scientific (Logan Utah), ICN Biomedicals, Inc. (Costa Mesa Calif.), Key Organics (Cornwall U.K.), Lancaster Synthesis (Windham N.H.), Maybridge Chemical Co. Ltd. (Cornwall U.K.), Parish Chemical Co. (Orem Utah), Pfaltz & Bauer, Inc. (Waterbury Conn.), Polyorganix (Houston Tex.), Pierce Chemical Co. (Rockford Ill.), Riedel de Haen AG (Hannover, Germany), Spectrum Quality Product, Inc. (New Brunswick, N.J.), TCI America (Portland Oreg.), Trans World Chemicals, Inc. (Rockville Md.), Wako Chemicals USA, Inc. (Richmond Va.), Novabiochem and Argonaut Technology, as well as from other API and pharmaceutical product manufacturers and distributors.

Compounds useful for co-administration with the PEGylated glutenase can also be made by methods known to one of ordinary skill in the art. As used herein, “methods known to one of ordinary skill in the art” may be identified though various reference books and databases. Suitable reference books and treatises that detail the synthesis of reactants useful in the preparation of compounds of the present invention, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Specific and analogous reactants may also be identified through the indices of known chemicals prepared by the Chemical Abstract Service of the American Chemical Society, which are available in most public and university libraries, as well as through on-line databases (the American Chemical Society, Washington, D.C., www.acs.org may be contacted for more details). Chemicals that are known but not commercially available in catalogs may be prepared by custom chemical synthesis houses, where many of the standard chemical supply houses (e.g., those listed above) provide custom synthesis services.

The PEGylated glutenase proteins of the invention and/or the compounds administered therewith can be incorporated into a variety of formulations for therapeutic administration provided by the present invention. In one aspect, the agents are formulated into pharmaceutical compositions by combination with appropriate, pharmaceutically acceptable carriers or diluents, and are formulated into preparations in solid, semi-solid, liquid or gaseous forms, such as tablets, capsules, powders, granules, ointments, solutions, suppositories, injections, inhalants, gels, microspheres, and aerosols. As such, administration of the PEGylated glutenase and/or other compounds can be achieved in various ways, although the route of administration is usually oral. The PEGylated glutenase and/or other compounds may in some instances act systemically after administration but more typically the site of drug action will be localized by virtue of the formulation, or by the use of an implant that acts to retain the API at the site of implantation.

In pharmaceutical dosage forms, the PEGylated glutenase and/or other compounds may be administered in the form of their pharmaceutically acceptable salts, or they may also be used alone or in appropriate association, as well as in combination with other pharmaceutically active compounds. The agents may be combined, as previously described, to provide a cocktail of activities. The following methods and excipients are exemplary and are not to be construed as limiting the invention.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

In one embodiment of the invention, the oral formulations comprise enteric coatings, so that the active agent, which could otherwise be degraded or inactivated in the stomach, is delivered in therapeutically effective amounts to the intestinal tract. A number of methods are available in the art for other drugs that can be modified as described herein to provide for the efficient delivery of enterically coated proteins into the small intestinal lumen. Most methods rely upon protein release as a result of the sudden rise of pH when food is released from the stomach into the duodenum, or upon the action of pancreatic proteases that are secreted into the duodenum when food enters the small intestine. For intestinal delivery of a PEP and/or a glutamine specific protease, the enzyme is usually lyophilized in the presence of appropriate buffers (e.g. phosphate, histidine, imidazole) and excipients (e.g. cryoprotectants such as sucrose, lactose, trehalose). Lyophilized enzyme cakes are blended with excipients, then filled into capsules, which are enterically coated with a polymeric coating that protects the protein from the acidic environment of the stomach, as well as from the action of pepsin in the stomach. Alternatively, protein microparticles can also be coated with a protective layer. Exemplary films are cellulose acetate phthalate, polyvinyl acetate phthalate, hydroxypropyl methylcellulose phthalate and hydroxypropyl methylcellulose acetate succinate, methacrylate copolymers, and cellulose acetate phthalate.

Other enteric formulations of the invention comprise engineered polymer microspheres made of biologically erodable polymers, which display strong adhesive interactions with gastrointestinal mucus and cellular linings and can traverse both the mucosal absorptive epithelium and the follicle-associated epithelium covering the lymphoid tissue of Peyer's patches. The polymers maintain contact with intestinal epithelium for extended periods of time and actually penetrate it, through and between cells. See, for example, Mathiowitz et al. (1997) Nature 386 (6623): 410-414. Drug delivery systems can also utilize a core of superporous hydrogels (SPH) and SPH composite (SPHC), as described by Dorkoosh et al. (2001) J Control Release 71(3):307-18.

Gluten detoxification for a gluten sensitive individual can commence as soon as food enters the stomach, because the acidic environment (˜pH 2) of the stomach favors gluten solubilization. Introduction of an acid-stable PEP or glutamine-specific protease into the stomach will synergize with the action of pepsin, leading to accelerated destruction of toxic peptides upon entry of gluten in the small intestines of celiac patients. In contrast to a PEP that acts in the small intestine, gastric enzymes need not be formulated with enteric coatings. Indeed, since several proteases (including the above-mentioned cysteine proteinase from barley) self-activate by cleaving the corresponding pro-proteins under acidic conditions. In one embodiment of the invention, the formulation comprises a pro-enzyme that is activated in the stomach.

Formulations are typically provided in a unit dosage form, where the term “unit dosage form,” refers to physically discrete units suitable as unitary dosages for human subjects, each unit containing a predetermined quantity of PEGylated glutenase in an amount calculated sufficient to produce the desired effect in association with a pharmaceutically acceptable diluent, carrier or vehicle. The specifications for the unit dosage forms of the present invention depend on the particular glutenase employed and the effect to be achieved with it, and the pharmacodynamics associated with the glutenase formulation in the host.

The pharmaceutically acceptable excipients, such as vehicles, adjuvants, carriers or diluents, are commercially available. Moreover, pharmaceutically acceptable auxiliary substances, such as pH adjusting and buffering agents, tonicity adjusting agents, stabilizers, wetting agents and the like, are commercially available. Any compound useful in the methods and compositions of the invention can be provided as a pharmaceutically acceptable base addition salt. “Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

Depending on the patient and condition being treated and on the administration route, the PEGylated glutenase may be administered in dosages of 0.01 mg to 500 mg/kg body weight per day, e.g. about 10, 20, 50, 100, 250, 500, 750 mg/day to 1, 2, 5, 10 or more g/day for an average person. Efficient proteolysis of gluten in vivo for an adult may, depending on diet and other factors, require at least about 500 units of a therapeutically efficacious PEP, In some embodiments, low dose PEP, such as 1000 units, can be used. In other embodiments, such as for the rapid detoxification of 5-10 g ingested gluten, as much as 20,000-50,000 units may be provided in unit dose form. One unit is defined as the amount of enzyme required to hydrolyze 1 μmol Cbz-Gly-Pro-pNA (for PEP) or Cbz-Gly-Gln-pNA (for a glutamine-specific protease) per min under specified conditions. Most PEPs have specific activities in the range of 5-50 units/mg protein. For barley EP-B2 (whose specific activity is in the 1000 Units/mg range, as measured with Cbz-Phe-Arg-pNA), low dose glutenase may consist of 10,000-100,000 Units, whereas high-dose glutenase contains as much as 1,000,000 Units. It will be understood by those of skill in the art that the dose can be raised, but that additional benefits may not be obtained by exceeding the useful dosage. Dosages can be appropriately adjusted for pediatric formulation. In children the effective dose may be lower, for example at least about 0.1 mg, or 0.5, 1, 10, 100, 250 to 750 mg/day, although in some embodiments the unit dose form administered to adults and children will be identical. In combination therapy involving, for example, a PEGylated PEP+DPP IV or PEGylated PEP+ DCP I, a comparable dose of the two enzymes may be given; however, the ratio will be influenced by the relative stability of the two enzymes toward gastric and duodenal inactivation and the desired site of action for each enzyme.

Enzyme treatment of Celiac Sprue is expected to be most efficacious when administered before or with meals. However, because food can reside in the stomach for 0.5-2 h, and for some formulations provided by the invention, the primary site of action is expected or desired to be in the small intestine, and the enzyme could also be administered after a meal, for example, within 0.5, to 1, to 2 hours after a meal.

Optimal gluten detoxification in vivo can also be achieved in accordance with the methods of the invention by combining an appropriate gastric active protease with a PEGylated PEP that acts upon gluten peptides in the duodenum, in concert with pancreatic enzymes. This can be achieved by co-administration of two enzyme doses, e.g. two capsules/tablets; via co-formulation of the two enzymes in appropriate quantities; and the like. Lyophilized duodenal PEGylated PEP particles or granules can be protected by a suitable polymeric enteric coating that promotes enzyme release only in the duodenum. In contrast, release of the gastric protease will be initiated immediately upon consumption of the dosage form. Combination therapies involving a PEGylated PEP and a complementary therapeutic agent, such as an inhibitor of the enzyme tissue transglutaminase, are also provided.

In some embodiments of the invention, the formulations provided comprise a cocktail of selected proteases. Such combinations of proteases may achieve a desired therapeutic effect more rapidly or economically than single protease formulations. In one combination formulation of the invention, PEGylated Flavobacterium PEP and Myxococcus PEP are co-formulated or co-administered, to allow for the destruction of a broader range of gluten antigenic peptides. In another combination, both PEPs in the formulation are PEGylated. Similarly, combination therapy with one or two PEGylated PEPs from the above list with an acid-stable PEP or glutamine endoprotease can lead to more gluten proteolysis in the stomach, thereby simplifying the task of gluten proteolysis in the upper small intestine.

In another embodiment, the formulation or administration protocol combines a PEGylated protease product and an inhibitor of transglutaminase 2 (TG2). Such formulations may have additional protection from gluten mediated enteropathy, as TG2 has been shown to have a significant pro-inflammatory effect on gluten peptides in the celiac gut. In particular, TG2 inhibitors containing halo-dihydroisoxazole, diazomethylketone or dioxoindole moieties are useful for this purpose. TG2 inhibitors described in, for example, US patent application publication Nos. US-2006-0035838-A1; US-2006-0052308; and U.S. provisional application Ser. No. 60/730,302 describe TG2 inhibitors useful in this method of the invention.

In another embodiment, the PEGylated protease or protease cocktail is administered and/or formulated with an anti-inflammatory agent, e.g. a statin; p38 MAP kinase inhibitor; anti-TNFα agent; or other similarly acting agent.

Those of skill will readily appreciate that dose levels can vary as a function of the specific enzyme, the severity of the symptoms and the susceptibility of the subject to side effects. Some of the PEGylated glutenases are more potent than others. Preferred dosages for a given enzyme are readily determinable by those of skill in the art by a variety of means in view of the disclosure herein. A preferred means is to measure the physiological potency of a given compound.

The therapeutic effect can be measured in terms of clinical outcome or can be determined by immunological or biochemical tests. Suppression of the deleterious T-cell activity can be measured by enumeration of reactive Th1 cells, by quantitating the release of cytokines at the sites of lesions, or using other assays for the presence of autoimmune T cells known in the art. Alternatively, one can look for a reduction in symptoms of a disease.

Various methods for administration may be employed, and the PEGylated proteins and pharmaceutical formulations will typically be administered orally, for example with meals. The dosage of the therapeutic formulation can vary widely, depending upon the nature of the disease, the frequency of administration, the manner of administration, the clearance of the agent from the host, and the like. For example, the initial dose can be larger, followed by smaller maintenance doses, or for example, the unit dose may vary depending on the amount of gluten to be ingested by the user, and the present invention provides unit dose forms of the PEGylated protein formulations to suit such varied needs. The dose can be administered as infrequently as weekly or biweekly, or more often fractionated into smaller doses and administered daily, with meals, semi-weekly, or otherwise as needed to maintain an effective dosage level. In one embodiment, the unit dose form is intended to be taken shortly before, during, or shortly after a meal in which the user expects to consume gluten. In such embodiments or others, the unit dose form may contain at least 10 mg of pegylated glutenase, where the weight is the protein weight prior to pegylation. In other embodiments, at least 100 mg, 250 mg, 500 mg or more of glutenase are in a unit dose, where the weight is the protein weight prior to pegylation. In one embodiment, sufficient glutenase to hydrolyze at least 1 g of gluten is in a unit dose. In other embodiments, sufficient glutenase is administered to hydrolyze 5 g, 10 g, 20 g or more gluten is in a unit dose.

Cross-reference to related applications. The present application is related to the following co-pending patent application which is filed on the same date on which the present application is filed, and which is incorporated herein in its entirety by reference: International patent application Ser. No. 06/______ entitled “Compositions and Methods for Enhanced Gastrointestinal Stability of Oligopeptides and Polypeptides” by Jonathon Gass (Attorney Docket ALVN-003WO), which claims priority to U.S. provisional application 60/725,733.

### EXPERIMENTAL

### PEGylation Leads to Improved Protease Resistance of Prolyl Endopeptidases

Prolyl endoptidases (PEPs) are serine proteases capable of hydrolyzing a peptide bond after an internal proline residue. Because of this unique specificity for proline residues, PEPs have been proposed as oral drug candidates to detoxify proline-rich, gluten-derived peptides that are toxic to Celiac Sprue patients (see, for example, PCT patent publication Nos. 2003/068170 and 2005/107786 and US patent application publication No. US-2006-0002917-A1. Celiac Sprue is an immune disorder of the small intestine that is triggered in response to dietary gluten, a protein mixture found in common foodgrains such as wheat, rye and barley. Gluten proteins are extremely rich in proline and glutamine residues, and the enteropathic response in Celiac Sprue patients is induced by presentation of proline-rich peptides derived from gluten by cleavage with gastric and pancreatic enzymes (pepsin, trypsin, chymotrypsin, elastase and carboxypeptidase). Unlike the enzymes normally present in the digestive tract, PEPs are capable of further cleaving these proline rich peptides in an endoproteolytic fashion. Encouragingly, pretreatment of gluten with PEPs lowers the gluten toxicity in Celiac Sprue patients.

A key challenge in formulating a PEP into an oral therapeutic agent for Celiac Sprue is to overcome its susceptibility to degradation by digestive proteases. The present invention shows that PEGylation of a bacterial PEP can significantly improve its proteolytic stability without detrimentally affecting the enzyme's activity or specificity.

### Methods

PEGylation reactions. Flavobacterium menningosepticum (FM) and Myxococcus xantus (MX) prolyl endopeptidases were expressed in E. coli and purified as previously described (see, for example, PCT patent publication No. 2005/107786). Activated PEGylating reagents were purchased from Nektar Therapeutics as succinimidyl propionate esters, which react with primary amine groups on the protein. Activated PEGs were obtained as mPEG-succinimidyl α-methylbutanoate (SMB) compounds with the following molecular weights: mPEG-SMB 2000 Da (Nektar 2M4K0D01), mPEG-SMP 5000 Da (2M4K0H01), mPEG-SMP 20,000 Da (Nektar 2M4K0P01) and mPEG-SMB 30,000 (Nektar 2M4K0R01).

Reactions were performed by mixing protein and activated PEGylating reagents, so that the ratio of PEG molecules to total number of lysine residues within a protein was 5:1. PEP was added to final protein concentration of 2 mg/mL. Reactions were carried out between 2 h and overnight at room temperature in PBS, pH 7.4. A control reaction consisted of only PEP in PBS.

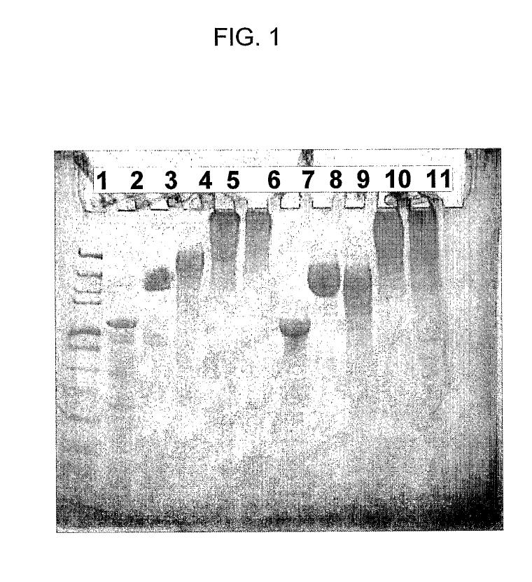

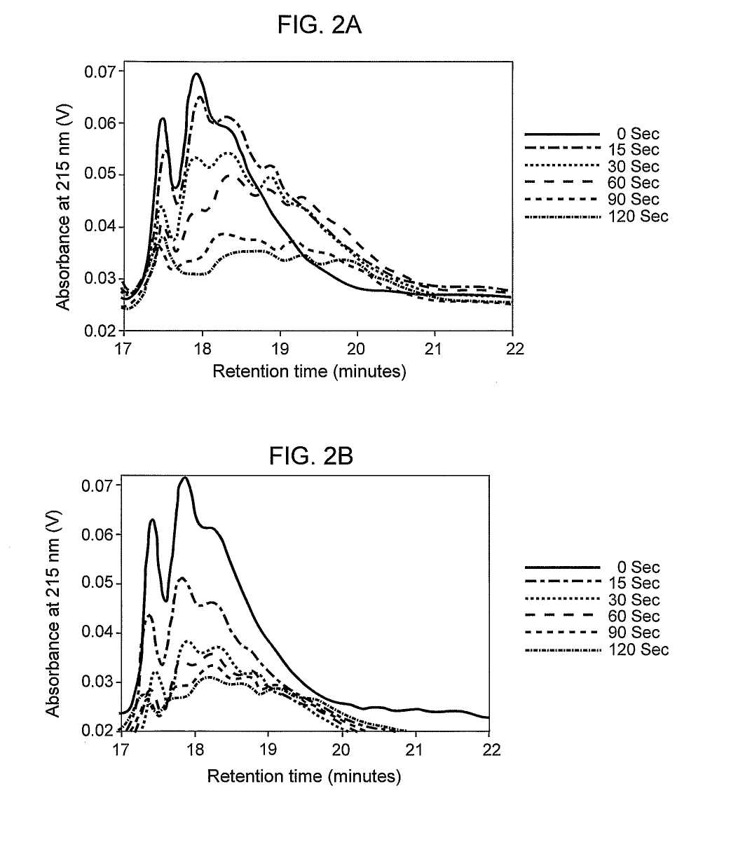

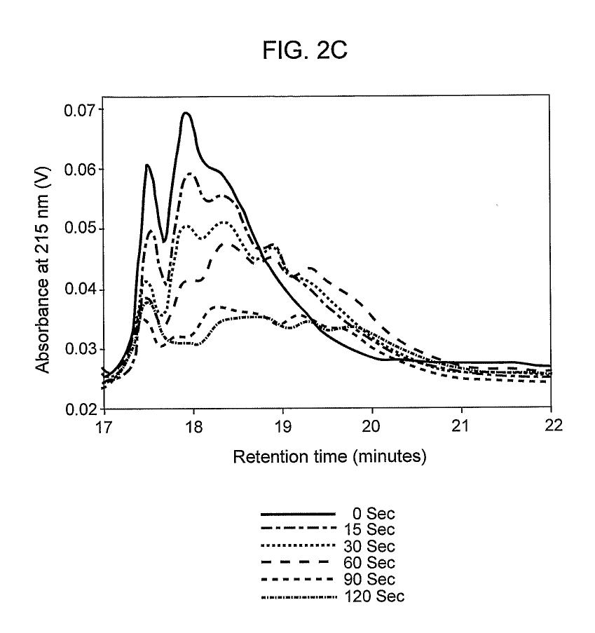

Products of PEGylation reactions were visualized on a 5-20% SDS-PAGE gel (LongLife Gels, Gradipore) (FIG. 1). Due to a high molecular weight of PEGylated proteins (100-200 kD), they could not be analyzed via standard electrospray mass spectrometry.

Cleavage of chromogenic substrates by PEGylated PEPs. 20 μg/mL of PEGylated protein (concentration based on protein-only weight, not on the weight of the modified enzyme) was added to 200 μl of 250 μM SucAlaPro-pNA in carbonate buffer (100 mM NaHCO3, 150 mM NaCl, pH 6.0). Reactions were carried out at room temperature. Release of p-nitroaniline (pNA) was monitored at 405 nm in 96-well plates, using a Molecular Devices Thermomax microplate reader at room temperature. Initial reaction rates were calculated from the slope of the A versus t plot during the first 2 min. Each sample was tested three times.

Cleavage of a long peptide substrate by PEGylated FM PEP. To demonstrate that the modified PEP is capable of processing longer, more pharmacologically relevant substrates, the enzyme was incubated with a 26mer peptide (FLQPQQPFPQQPQQP YPQQPQQPFPQ), derived from γ-gliadin, a constituent of wheat gluten. 30 uM of the 26mer peptide was incubated with 0.5 uM of FM PEP, in carbonate buffer (the modified) was added to 200 ul of 250 uM SucAlaProPNA in carbonate buffer (100 mM NaHCO3, 150 mM NaCl, pH 6.0) at 37° C., in a water bath. The reaction was quenched at various time points (0, 15, 30, 60, 90 and 120 seconds) by adding 5% TFA to a final concentration of 0.5% TFA. Samples were analyzed on a 4.6×150 mm reverse phase C-18 protein & peptide column (Vydac, Hesperia) using Rainin Dynamax SD-200 pumps (1 ml/min), a Varian 340 UV detector set at 215 nm and a Varian Prostar 430 autosampler. Solvent A was water with 5% acetonitrile and 0.1% TFA. Solvent B was acetonitrile with 5% water and 0.1% TFA. Prior to injection, samples were filtered through a 0.2 μm, low protein binding affinity filter.

Trypsin and chymotrypsin stability of PEGylated PEPs. To demonstrate how PEGylation affects the protease-resistance profile of FM PEP, degradation of PEP by pancreatic proteases trypsin and chymotrypsin was analyzed under various substrate and enzyme concentrations.

To demonstrate how susceptible PEP and PEGylated PEP are to cleavage by trypsin, the PEP activity remaining after 5 minutes of incubation with various levels of trypsin and chymotrypsin was measured. 0.6 μM of unmodified or PEGylated FM PEP or MX PEP were reacted with excess of trypsin or chymotrypsin (100, 200 and 400 μM) in carbonate buffer at room temperature. PEP activity remaining after 5 minutes was assayed against chromogenic substrate (as described above), and compared to PEP activity prior to incubation with trypsin and chymotrypsin.

To demonstrate how the rate of cleavage of FM PEP by trypsin and chymotrypsin depends on the substrate concentration (FM PEP), 25 μM of trypsin or chymotrypsin were incubated with various concentrations (from 0.6 to 13 μM) of FM PEP. Incubations were carried out at room temperature for various lengths of time (between 0 and 40 min). Residual PEP activity after incubation with a pancreatic protease was assayed using chromogenic substrate as described above. The rate of PEP deactivation was fitted using the best linear fit (SigmaPlot), and the resulting rates were plotted as a function of PEP concentration (FIG. 4). The dependence of initial rates of cleavage on concentration of PEP was fitted to the Michaelis-Menten equation using SigmaPlot.

Prolyl endopeptidases can be modified with activated PEGs of various molecular weights. Reaction of certain activated PEGs with protein depends on the availability of and reactivity of lysines on the protein surface. FM PEP has a total of 71 lysines, whereas MX PEP has a total of 44 lysine residues. Analysis of the MX PEP crystal structure revealed that approximately 50% of lysines (24 residues) lie on the surface of the protein. If one assumes that only these recognition sites were reactive, then the FM PEP was modified with 10-fold excess PEGylating reagent relative to potential lysine conjugation sites. Gel shift analysis via SDS PAGE showed that regardless of the molecular weight of active PEGylating reagents used for conjugation, both PEPs were completely modified within two hours (FIG. 1). Even though it was not possible to determine the exact molecular weight of the protein-PEG conjugate by SDS PAGE or electrospray mass spectrometry, the molecular masses of all PEGylation products were considerably higher than those of the unmodified protein. Based on the relative migration of the PEGylated products and the MW markers, approximately 20 lysines were modified by PEG2000, 10 lysines were modified by PEG5000, and at least 10 lysines were modified by PEG 20,000 and PEG 30,000.

PEGylated PEPs are enzymatically active. Despite the extensive modification of protein surface, PEGylation with 5,000, 20,000 and 30,000 Da PEGs did not have a negative effect on post-proline cleaving ability of FM PEP, as determined by a chromogenic assay, using SucAlaPro-pNA as a substrate (Table 1).

TABLE 1
Prolyl endopeptidase activity assays on small
chromogenic substrate, SucAlaProPNA.

| | unmodi- | | | | |
|---|---|---|---|---|---|
| | fied | PEG-2000 | PEG-5000 | PEG-20,000 | PEG-30,000 |
| FM | 100% | 44 ± 14% | 307 ± 40% | 114 ± 30% | 232 ± 35% |
| MX | 100% | 64 ± 20% | 170 ± 30% | 220 ± 30% | 320 ± 50% |

Numbers represent percent specific activity relative to the specific activity of corresponding unmodified PEP.

In fact, Table 1 shows these PEGylated PEPs were more active against the chromogenic substrate than the unmodified proteins. In contrast to these longer-chain PEG reagents, PEGylation with a 2000-Da PEG reagent resulted in partial deactivation of both FM and MX PEP. PEG 2000, is relatively small compared to other PEGs tested, and it is possible that it reacts with a more internal lysine, thus interfering with the enzyme active site. Other glutenases may not be partially inactivated under these PEGylation conditions, and PEG 2000 may not inactivate FM and/or MX PEP to any significant extent under other coupling conditions.

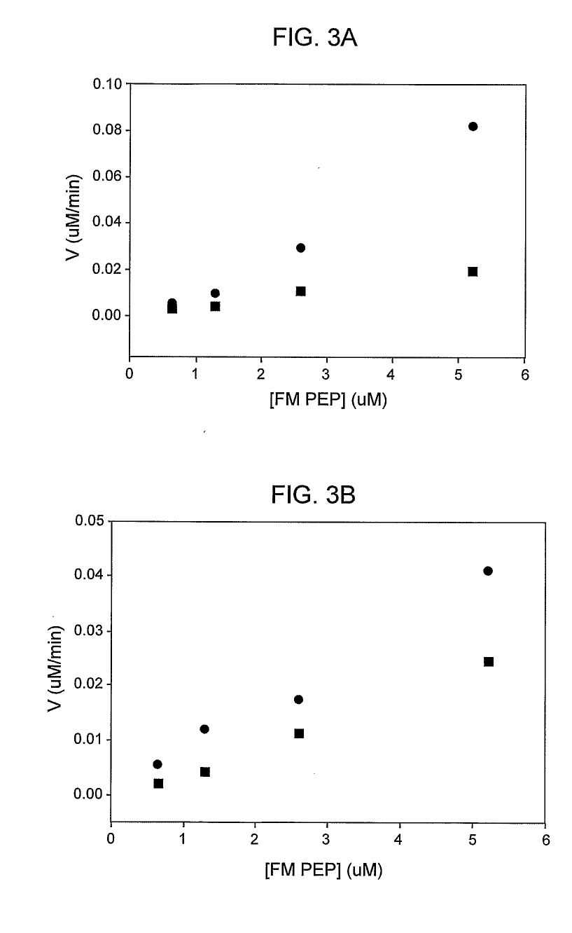

PEGylated enzymes are able to cleave longer substrates at rates comparable to unmodified enzyme. The time course of cleavage of a long, gluten-derived 26mer peptide was monitored via HPLC, and showed that PEGylated enzymes maintain their specificity for longer substrates (FIG. 3). Disappearance of the peak corresponding to the intact 26mer was quantified using numerical integration. Interestingly, both the 5 kDa and 20 kDa PEGylated FM PEPs were 8-12% faster in cleaving the peptide than the unmodified FM PEP. This is consistent with increased rate of cleavage of the chromogenic substrate by PEGylated PEPs. In one embodiment, the invention provides a PEGylated glutenase that cleaves a gluten peptide faster than the corresponding non-PEGylated glutenase.

PEGylated enzymes of the invention have increased resistance to cleavage by trypsin and chymotrypsin. At physiological trypsin and chymotrypsin concentrations (between 2.5 and 5 mg/mL), PEGylated forms of FM PEP were less susceptible to trypsin and chymotrypsin degradation, compared to the native unmodified protein (Table 2). For example, after a 5 min incubation with 5 mg/mL trypsin at pH 6, there was approximately 6 times more PEP activity remaining in a reaction containing FM PEP modified with PEG-5000 as compared to unmodified FM PEP (56% activity remaining relative to starting FM5000 activity * 3-fold increase in PEP activity due to PEGylation/27% PEP activity remaining for unmodified protein). PEGylated PEPs (FM PEG-5000, FM PEG 20,000 and FM PEG-30,000) were more active to start with, and the larger percent of their activity was retained after incubation with trypsin and chymotrypsin compared to the unmodified PEP. This resulted in a significant increase of PEP available after a 5 min incubation with trypsin or chymotrypsin.

TABLE 2
FM PEP activity remaining after a 5 min incubation with various
concentrations of trypsin (a) and chymotrypsin (b).
a)

Trypsin concentration

| | 2.5 mg/mL | 5.0 mg/mL |
|---|---|---|
| FM0 | 52% | 27% |
| FM5k | 83% | 56% |
| FM20k | 81% | 52% |
| FM30k | 73% | 43% |

b)

Chymotrypsin concentration

| | 2.5 mg/mL | 5.0 mg/mL |
|---|---|---|
| FM0 | 74% | 18% |
| FM5k | 100% | 76% |
| FM20k | 100% | 79% |
| FM30k | 100% | 81% |

Numbers represent PEP activity detected after 5 minutes relative to activity prior to incubation with trypsin or chymotrypsin. Note that the starting activity levels are different for each PEGylated PEP species, hence to obtain the total enzymatic activity after trypsin incubation, these percentages should be scaled up with the increase of activity due to PEGylation (see Table 1).

The dependence of initial reaction rates of trypsin and chymotrypsin-catalyzed proteolysis of PEP on the substrate concentration showed that, at all substrate concentrations examined, unmodified PEP was a better substrate for both trypsin and chymotrypsin. Fitting the trypsin cleavage data to Michaelis Menten equation, a k_{cat}/K_{M} of 11.7 M⁻¹ sec-1 was obtained for unmodified PEP, and a k_{cat}/K_{M} of 3.4 was obtained for the PEGylated PEP. The chymotrypsin proteolysis exhibited a k_{cat}/K_{M} of 5.0 for the unmodified PEP, and 3.3 for the PEGylated enzyme.

Development of a PEP-based treatment for Celiac Sprue depends on the ability of such a drug candidate to efficiently cleave and detoxify multiple gluten-derived peptides. If this process is to occur in vivo, then it has has to occur in the complex, protease-rich environment of the digestive tract to and potentially through the upper small intestine. Cleavage of gluten-based peptides by PEP must occur in concert with normal proteolytic activity of the body at the site at which cleavage occurs, such as the stomach or duodenum. Because a PEP is itself a substrate of pancreatic and other digestive enzymes in the human gut, the goal is to ensure that this complex set of proteolysis reactions results in the greatest possible reduction of immunotoxic gluten peptide concentrations before the PEP is fully degraded by pancreatic and other digestive enzymes enzymes.

PEGylation reactions yielded homogenous and enzymatically active PEP. Unexpectedly, it was found that PEGylated PEPs can exhibit increased specific activity compared to an unmodified PEP. PEGylation may have an effect on the molecular dynamics of PEP protein, resulting in a slight improvement in the active site stereochemistry.

One concern with extensive modification of protein surface is that the modified protein could lose its ability to process large substrates. This aspect of activity is particularly important for a cleavage of large peptide fragments involved in the pathogenesis of Celiac Sprue. Demonstrating that the PEGylated PEP cleaves a 26-amino acid long, gluten-derived peptide, previously shown to elicit a T-cell response associated with Celiac Sprue, demonstrates that PEGylated enzymes can retain specificity for larger substrates. The PEGylated enzymes used in the demonstration were actually slightly better at cleaving this substrate compared to the unmodified enzyme, suggesting that molecular motions involved in substrate processing are not hindered by modification of protein surface.

In addition to maintaining the normal activity and specificity of PEP, the PEGylated PEP was better able to withstand proteolysis by trypsin and chymotrypsin. Interestingly, chymotrypsin cleavage was also inhibited by PEGylation, even though the residues cleaved were not modified themselves. This shows that PEGylation of lysines has a significant effect on the whole protein surface, and PEGylated PEPs may be demonstrated to have improved resistance to other proteases as well.

In summary, PEGylation of PEP yields an improved glutenase for detoxification of gluten-derived peptides for treatment of Celiac Sprue under physiologically relevant conditions. Chemical modification of a PEP by PEGylation can improve the gluten-detoxification profile of the PEP.

The following examples provide those of ordinary skill in the art with a complete disclosure and description of how to make and use certain illustrative embodiments of the present invention, and are not intended to limit the scope of the invention or to represent that the experiments below are all or the only experiments performed. Efforts have been made to ensure accuracy with respect to numbers used (e.g., amounts, temperature, and the like), but some experimental errors and deviations may be present. Unless indicated otherwise, parts are parts by weight, molecular weight is weight average molecular weight, temperature is in degrees Centigrade, and pressure is at or near atmospheric.

All publications, patents, and patent applications cited in this specification are herein incorporated by reference as if each individual publication, patent, or patent application were specifically and individually indicated to be incorporated by reference.

The present invention has been described in terms of particular embodiments found or proposed by the inventor to comprise preferred modes for the practice of the invention. It will be appreciated by those of skill in the art that, in light of the present disclosure, numerous modifications and changes can be made in the particular embodiments exemplified without departing from the intended scope of the invention. Moreover, due to biological functional equivalency considerations, changes can be made in methods, structures, and compounds without affecting the biological action in kind or amount. All such modifications are intended to be included within the scope of the appended claims.

1. An isolated, biologically active glutenase conjugated to at least one polyethylene glycol (PEG) moiety.

2. The isolated, biologically active glutenase of claim 1 that is a prolyl endopeptidase conjugated to at least one polyethylene glycol (PEG) moiety.

3. The prolyl endopeptidase of claim 2, wherein said PEG moiety is at least 2000 Da.

4. The prolyl endopeptidase of claim 2, wherein said PEG moiety is at least 5000 Da.

5. The prolyl endopeptidase of claim 4, wherein said said prolyl endopeptidase is Flavobacterium meningosepticum PEP; Myxococcus xanthus PEP; Sphingomonas capsulata PEP; Lactobacillus helveticus PEP; Aspergillus niger PEP or Penicillium citrinum PEP.

6. A pharmaceutical formulation, comprising: an effective dose of the prolyl endopeptidase of claim 1; and a pharmaceutically acceptable excipient.

7. The formulation according to claim 6, wherein said formulation is suitable for oral administration.

8. The formulation according to claim 6, wherein said formulation comprises an enteric coating.

9. A method of treating Celiac Sprue and/or dermatitis herpetiformis, the method comprising:
administering to a patient an effective dose of a glutenase according to claim 1; wherein said glutenase attenuates gluten toxicity in said patient.
